(19) United States
(12) Patent Application Publication
(10) Pub. No.: US 2025/0283043 A1
(43) Pub. Date: Sep. 11, 2025

(54) METHODS FOR DIFFERENTIATION OF INDUCED PLURIPOTENT STEM CELLS USING HOLLOW FIBER BIOREACTORS

(71) Applicant: Terumo BCT, Inc., Lakewood, CO (US)

(72) Inventors: Nathan D. FRANK, Arvada, CO (US); Mindy M. MILLER, Lakewood, CO (US)

(21) Appl. No.: 19/059,507

(22) Filed: Feb. 21, 2025

Related U.S. Application Data
(60) Provisional application No. 63/563,686, filed on Mar. 11, 2024.

(51) Int. Cl. C12N 5/0783; C12M 1/12; C12M 3/06

(52) U.S. Cl. CPC C12N 5/0646; C12M 25/10; C12M 27/14; C12N 2506/45

(57) ABSTRACT

A method of differentiating induced pluripotent stem cells using a hollow fiber bioreactor includes obtaining induced pluripotent stem cells in the hollow fiber bioreactor and causing a differentiating medium to move through the hollow fiber bioreactor. In certain variations, the obtaining of the induced pluripotent stem cells may include expanding the induced pluripotent stem cell using the hollow fiber bioreactor. In other variations, the obtaining of the induced pluripotent stem cells may include introducing the induced pluripotent stem cells into the hollow fiber bioreactor.

### CROSS-REFERENCE TO RELATED APPLICATIONS

This application claims the benefit of U.S. Provisional Application No. 63/563,686 filed on Mar. 11, 2024. The entire disclosure of the above application is incorporated herein by reference.

### FIELD

The present disclosure relates to methods for differentiation of induced pluripotent stem cells using hollow fiber bioreactors.

### BACKGROUND

This section provides background information related to the present disclosure which is not necessarily prior art.

Induced pluripotent stem cells (iPSCs) are cells that are derived from a range of cells from terminally differentiated tissues that have been induced to a pluripotent state. Differentiation includes exposing the cell population including the induced pluripotent stem cells to particular compounds that trigger the development of the induced pluripotent stem cells into any terminally differentiated cell type, including, for example, natural killer cells, cardiomyocytes, mesenchymal stem cells, T cells, or any combination thereof.

### SUMMARY

This section provides a general summary of the disclosure and is not a comprehensive disclosure of its full scope or all of its features.

In various aspects, the present disclosure provides a method of differentiating induced pluripotent stem cells using a hollow fiber bioreactor.

In at least one example embodiment, the method may include obtaining induced pluripotent stem cells in the hollow fiber bioreactor and causing a differentiating medium to move through the hollow fiber bioreactor.

In at least one example embodiment, the obtaining of the induced pluripotent stem cells may include expanding the induced pluripotent stem cell using the hollow fiber bioreactor.

In at least one example embodiment, the obtaining of the induced pluripotent stem cells may include introducing the induced pluripotent stem cells into the hollow fiber bioreactor.

In various aspects, the present disclosure provides a method of preparing natural killer cells using a hollow fiber bioreactor.

In at least one example embodiment, the method may include obtaining induced pluripotent stem cells, differentiating the induced pluripotent stem cells to CD34+ cells using the hollow fiber bioreactor, and differentiating the CD34+ cells to natural killer cells using the hollow fiber bioreactor.

In at least one example embodiment, the obtaining of the induced pluripotent stem cells may include expanding the induced pluripotent stem cell using the hollow fiber bioreactor.

In at least one example embodiment, the obtaining of the induced pluripotent stem cells may include introducing the induced pluripotent stem cells into the hollow fiber bioreactor.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1 is an illustration of an example cell expansion system having a bioreactor in accordance with at least one example embodiment of the present disclosure;

FIG. 2 is an illustration of an example bioreactor that shows circulation paths through the bioreactor, and which may be incorporated into cell expansion systems such as the cell expansion system illustrated in FIG. 1, in accordance with at least one example embodiment of the present disclosure;

FIG. 3 is an illustration of another example bioreactor that shows circulation paths through the bioreactor, and which may be incorporated into cell expansion systems such as the cell expansion system illustrated in FIG. 1, in accordance with at least one example embodiment of the present disclosure;

FIG. 4 is an illustration of an example rocking device configured to move a bioreactor, such as the bioreactor of FIG. 2 or FIG. 3, in accordance with at least one example embodiment of the present disclosure;

FIG. 5 is a schematic illustrating example flow paths of an example cell expansion system, like the cell expansion system illustrated in FIG. 1, in accordance with at least one example embodiment of the present disclosure; and

FIG. 6 is a flowchart illustrating an example method for differentiating a cell population including induced pluripotent stem cells using an example cell expansion system, like the cell expansion system illustrated in FIG. 1, to form natural killer cells in accordance with at least one example embodiment of the present disclosure.

FIG. 7 is a flowchart illustrating an example method for differentiating a cell population including induced pluripotent stem cells using an example cell expansion system, like the cell expansion system illustrated in FIG. 1, in accordance with at least one example embodiment of the present disclosure.

Corresponding reference numerals indicate corresponding parts throughout the several views of the drawings.

### DETAILED DESCRIPTION

Example embodiments will now be described more fully with reference to the accompanying drawings.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected, or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers, and/or sections, these elements, components, regions, layers, and/or sections should not be limited by these terms. These terms may be only used to distinguish one element, component, region, layer or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

Various components are referred to herein as “operably associated.” As used herein, “operably associated” refers to components that are linked together in operable fashion and encompasses embodiments in which components are linked directly, as well as embodiments in which additional components are placed between the linked components. “Operably associated” components can be “fluidly associated.” “Fluidly associated” refers to components that are linked together such that fluid can be transported between them. “Fluidly associated” encompasses embodiments in which additional components are disposed between the two fluidly associated components, as well as components that are directly connected. Fluidly associated components can include components that do not contact fluid but contact other components to manipulate the system (e.g., a peristaltic pump that pumps fluids through flexible tubing by compressing the exterior of the tube).

In this application, including the definitions below, the term “module” or the term “controller” may be replaced with the term “circuit.” The term “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

The term code, as used above, may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The term memory circuit is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory, tangible computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general-purpose computer to execute one or more particular functions embodied in computer programs. The functional blocks, flowchart components, and other elements described above serve as software specifications, which can be translated into the computer programs by the routine work of a skilled technician or programmer.

The computer programs include processor-executable instructions that are stored on at least one non-transitory, tangible computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation) (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Swift, Haskell, Go, SQL, R, Lisp, Java®, Fortran, Perl, Pascal, Curl, OCaml, JavaScript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

Example embodiments will now be described more fully with reference to the accompanying drawings.

The present disclosure relates to methods for differentiation of induced pluripotent stem cells using bioreactor-based systems like those described in U.S. application Ser. No. 15/943,536 as filed Apr. 2, 2018 and titled EXPANDING CELLS IN A BIOREACTOR, which published as U.S. Pub. No. 2018/0282695 on Oct. 2, 2018; U.S. Pat. No. 10,577,585 as issued on Mar. 3, 2020 and titled CELL EXPANSION, which was filed as U.S. application Ser. No. 15/153,396 on May 12, 2016; U.S. Pat. No. 11,685,883 as issued on Jun. 27, 2023 and titled METHODS AND SYSTEMS FOR COATING A CELL GROWTH SURFACE, which was filed as U.S. application Ser. No. 15/616,635 on Jun. 7, 2017; U.S. application Ser. No. 17/702,658 as filed Mar. 23, 2022 and titled CELL CAPTURE AND EXPANSION, which published as U.S. Pub. No. 2022/0306978 on Sep. 29, 2022; U.S. application Ser. No. 16/845,686 as filed Apr. 10, 2022 and titled METHODS AND SYSTEMS FOR COATING A CELL GROWTH SURFACE, which published as U.S. Pub. No. 2020/0239819 on Jul. 30, 2020; U.S. application Ser. No. 17/087,571 as filed Nov. 2, 2022 and titled CELL EXPANSION, which published as U.S. Pub. No. 2021/0047602 on Feb. 18, 2021; U.S. Pat. No. 11,702,634 as issued Jul. 18, 2023 and titled EXPANDING CELLS IN A BIOREACTOR, which was filed as U.S. application Ser. No. 15/943,536 on Apr. 2, 2018; U.S. application Ser. No. 18/234,470 as filed Aug. 16, 2023 and titled METHODS FOR CELL EXPANSION, DIFFERENTIATION, AND/OR HARVESTING OF NATURAL KILLER CELLS USING HOLLOW-FIBER MEMBRANES; and U.S. App. No. 63/407,987 as filed Sep. 19, 2022 and titled EXPANDING CELLS, the entire disclosures of which are hereby incorporated by reference.

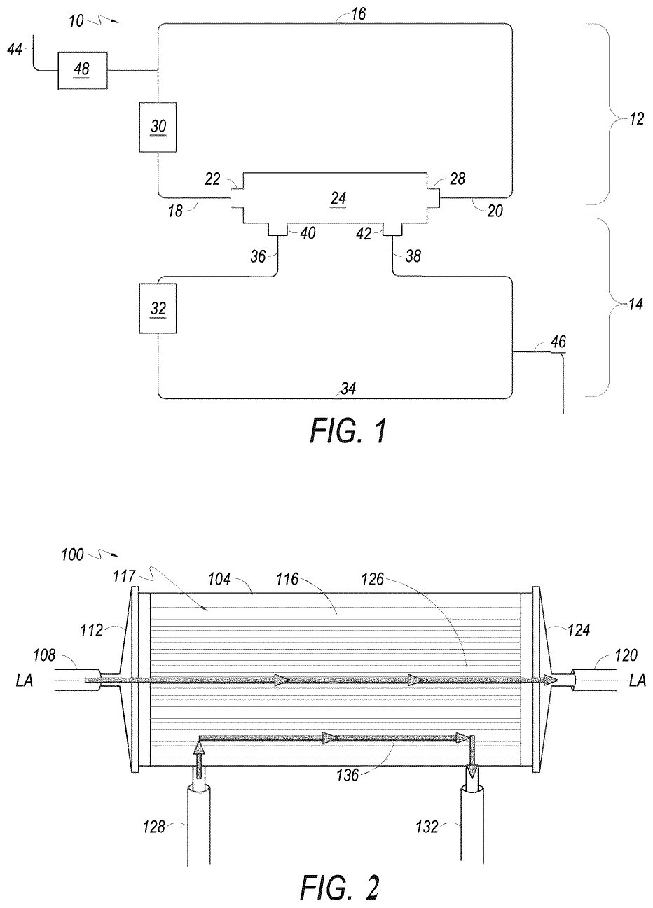

FIG. 1 illustrates an example cell expansion system 10 that includes a first fluid circulation path 12 and a second fluid circulation path 14. The first fluid circulation path 12 includes, for example, a first fluid flow path 16 having opposing ends 18 and 20. The first fluid flow path 16 may be in fluid communication with a hollow fiber cell growth chamber 24 (which can also be referred to as a “bioreactor”). For example, the first opposing end 18 of the first fluid flow path 16 may be in fluid communication with a first inlet 22 of the cell growth chamber 24, and the second opposing end 20 may be in fluid communication with first outlet 28 of the cell growth chamber 24. Fluid in the first circulation path 12 may flow through an interior of a plurality of hollow fibers 116 of a hollow fiber membrane (“HFM”) 117 (see, e.g., FIG. 2) disposed in the cell growth chamber 24. In at least one example embodiment, a first fluid flow control device 30 may be operably coupled to the first fluid flow path 16 and may control the flow of fluid in first fluid circulation path 12.

The second fluid circulation path 14 includes, for example, a second fluid flow path 34 and a second fluid flow control device 32. Like the first fluid flow path 16, the second fluid flow path 34 may have opposing ends 36 and 38. The opposing ends 36 and 38 of second fluid flow path 34 may in fluid communication with an inlet port 40 and an outlet port 42 of the cell growth chamber 24. For example, a first opposing end 36 of the second fluid flow path 34 may be in fluid communication with the inlet port 40 of the cell growth chamber 24, and the second opposing end 38 of the second fluid flow path 34 may be in fluid communication with the outlet port 42. Fluid in the second circulation path 14 may be in contact with an outside of the hollow fiber membrane 117 (see, e.g., FIG. 2) disposed in the cell growth chamber 24. In at least one example embodiment, a second fluid flow control device 32 may be operably coupled to the second fluid flow path 34 and may control the flow of fluid in the second fluid circulation path 14.

The first and second fluid circulation paths 12, 14 may be maintained in the cell growth chamber 24 by way of the hollow fiber membrane 117, where fluid in first fluid circulation path 12 flows through an intracapillary (“IC”) space of the hollow fiber membrane 117 and fluid in the second circulation path 14 flows through the extracapillary (“EC”) space of the cell growth chamber 24. The first circulation path 12 may be referred to as the “intracapillary loop” or “IC loop”. The second fluid circulation path 14 may be referred to as the “extracapillary loop” or “EC loop”. Fluid in first fluid circulation path 12 may flow in either a co-current or counter-current direction with respect to a fluid flow in second fluid circulation path 14.

In at least one example embodiment, a fluid inlet path 44 may be fluidly associated with the first fluid circulation path 12, and a fluid outlet path 46 may be fluidly associated with the second fluid circulation path 14. The fluid inlet path 44 permits fluid into first fluid circulation path 12, while the fluid outlet path 46 permits fluid to exit the cell expansion system 10. In at least one example embodiment, as illustrated, a third fluid flow control device 48 may be operably associated with the fluid inlet path 44. Although not illustrated, it should be recognized that, in various other example embodiments, a fourth fluid flow control device may alternatively or additionally be associated operably associated with the first outlet path 46. In at least one example embodiment, the fluid flow control devices (including the first fluid flow control device 30, the second fluid flow control device 32, the third fluid flow control device 48, and/or the fourth fluid flow control device) may include a pump, valve, clamp, or any combination thereof. For example, multiple pumps, valves, and clamps can be arranged in any combination. In at least one example embodiment, the fluid flow control device may include a peristaltic pump. Fluid circulation paths (including the first fluid circulation path 12 and/or the second fluid circulation path 14), inlet ports (including the fluid inlet port 44), and/or the outlet port (including the fluid outlet port 46), may include any known tubing material, and any kind of fluid-including, for example, buffers, protein containing fluid, and cell-containing fluid-can flow through the various circulation paths (including the first fluid circulation path 12 and/or the second fluid circulation path 14), inlet paths (including the fluid inlet port 44), and outlet paths (including the fluid outlet port 46). It should be recognized that the terms “fluid,” “media,” and “fluid media” are used interchangeably.

An example hollow fiber cell growth chamber 100 (which can also be referred to as a “bioreactor”) is illustrated in FIG. 2. The hollow fiber cell growth chamber 100 may be used as the hollow fiber cell growth chamber 24 of the cell expansion system 10 illustrated in FIG. 1. The hollow fiber cell growth chamber 100 has a longitudinal axis (represented by the line LA-LA) and includes a cell growth chamber housing 104. The cell growth chamber housing 104 may have four openings or ports, including, for example, an intracapillary inlet port 108, an intracapillary outlet port 120, an extracapillary inlet port 128, and an extracapillary outlet port 132. A first fluid (which can also be referred to as an intracapillary fluid or media) in a first circulation path (such as the first fluid circulation path 12) can enter the cell growth chamber 100 through the intracapillary inlet port 108 at a first fluid manifold end 112 of the cell growth chamber 100 and into and through the intracapillary spaces of a plurality of hollow fibers 116 and out of cell growth chamber 100 through intracapillary outlet port 120, which is located at a second fluid manifold end 124 of the cell growth chamber 100. The fluid path between the intracapillary inlet port 108 and the intracapillary outlet port 120 defines an intracapillary portion 126 of the cell growth chamber 100. A second fluid (which can also be referred to as an extracapillary media or fluid) in a second circulation path (such as the second fluid circulation path 14) can enter the cell growth chamber 100 through the extracapillary inlet port 128. This second fluid contacts the extracapillary space or outside of the hollow fiber membrane 117 and exits the cell growth chamber 100 via the extracapillary outlet port 132. The fluid path between the extracapillary inlet port 128 and the extracapillary outlet port 132 defines an extracapillary portion 136 of the cell growth chamber 100.

Often, cells (for example, induced pluripotent stem cells) are seeded (for example, for expansion, differentiation, and/or harvesting) in the intracapillary portion 126, while a cell culture medium is pumped through the extracapillary portion 136 to deliver nutrients to the cells via hollow fiber membrane perfusion during expansion. However, in other variations, cells (for example, induced pluripotent stem cells) can be seeded (for example, for expansion, differentiation, and/or harvesting) in the extracapillary portion 136, while the cell culture medium is pumped through the intracapillary portion 126 to deliver nutrients to the cells via hollow fiber membrane perfusion during expansion. In still further variations, cells (for example, induced pluripotent stem cells) may be seeded (for example, for expansion, differentiation, and/or harvesting) in the intracapillary portion 126, while the cell culture medium is pumped through both the extracapillary portion 136 and the intracapillary portion 126. In such instances, movement of the cell culture medium through the intracapillary portion 126 can help to remove excess cells not adhered to surfaces of the hollow fiber membrane. In each instance, the material used to form the hollow fiber membrane 117 may be any biocompatible polymeric material that is capable of being made into the hollow fiber membrane 117. For example, synthetic polysulfone-based materials (e.g., polyethersulfones (PES)) are often used to form the hollow fiber membrane 117.

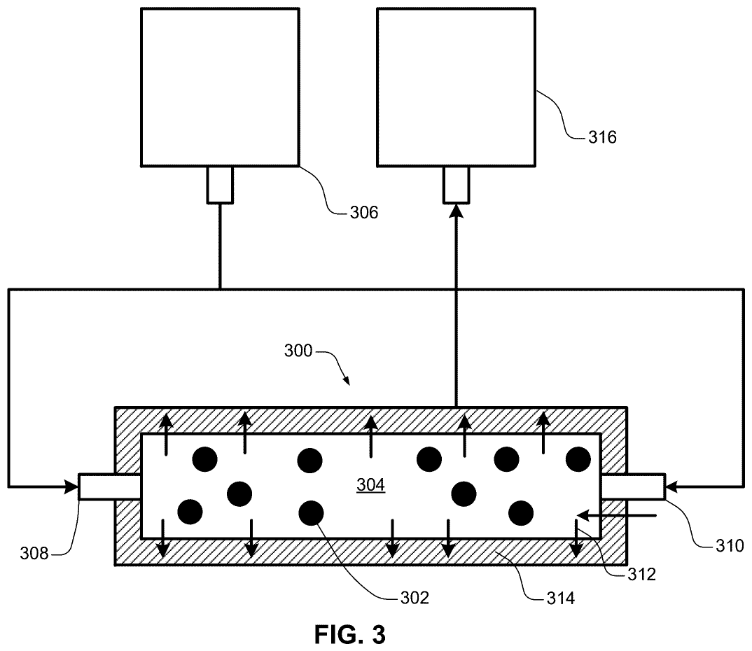

FIG. 3 provides another example of a hollow fiber cell growth chamber 300 (which can also be referred to as a “bioreactor”). The hollow fiber cell growth chamber 300 may a schematic of example counterflow containment of cells. As illustrated, cells 302 may be positioned (or repositioned) within an intracapillary portion or space 304 of the hollow fiber cell growth chamber 300 using counterflow containment. More specifically, as media or fluid moves (i.e., flows) from a media bag or container 306 it may be split between an intracapillary inlet pump (not shown) and an intracapillary circulation pump (not shown) such that the media or fluid moves into the intracapillary portion or space 304 of the hollow fiber cell growth chamber 300 through both an intracapillary inlet 308 and an intracapillary outlet 310. In contrast, cells 302 may be seeded and/or recirculated using unidirectional flow from the media bag or container 306 to the intracapillary portion or space 304 of the hollow fiber cell growth chamber 300 via the intracapillary inlet 308. The cells 302 may be a cell type that has been expanded in quantum/quantum flex. For example, the cells 302 may be iPSCs, Mesenchymal stem cells (MSCs), Tcells, natural killer (NK) cells, human embryonic kidney 293 (HEK293) cells, and/or Jurkat cells.

In at least one example embodiment, during a feeding stage, the intracapillary inlet flow rate is about 2 times of the intracapillary circulation pump rate. For example, the intracapillary inlet flow rate may be greater than or equal to about 0.02 mL/min and the intracapillary circulation pump rate may be greater than or equal to about 0.01 mL/min. Arrows 312 illustrate the fluid movement through pores of the hollow fiber cell growth chamber 300 from the intracapillary portion or space 304 to the extracapillary portion or space 314 during counterflow containment. A waste bag 316 may be in fluid communication with the extracapillary portion or space 314 allowing appropriate movement from the extracapillary portion 314 to the waste bag 316.

As discussed above, although the cells 302 are illustrated as being seeded within the intracapillary portion or space 304, it should be appreciated that, in various other example embodiments, the cells 302 may instead be seeded instead in the extracapillary portion or space 314. In such instances counterflow containment may include moving media or fluid from the media bag or container 306 to an extracapillary inlet (not shown) and also an extracapillary outlet (not shown).

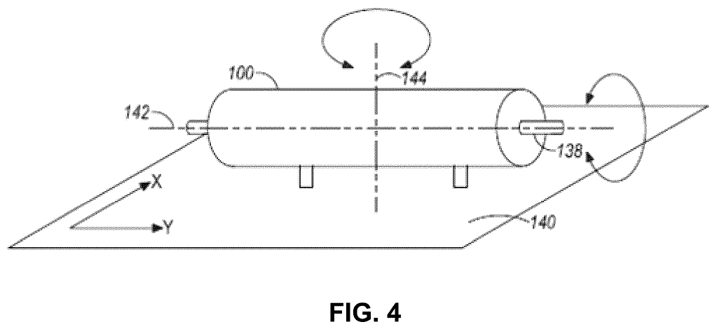

In at least one example embodiment, the cell expansion system 10 may also include a device that is configured to move or “rock” the cell growth chamber 100 relative to other components of the cell expansion system 10. The device may be a rotational and/or lateral rocking device. For example, as illustrated in FIG. 4, the cell growth chamber 100 may be rotationally connected to one or more rotational rocking components 138 and to a lateral rocking component 140. A first rotational rocking component 138 may be rotationally associated with the bioreactor 100. For example, the first rotational rocking component 138 may be configured to rotate the bioreactor 100 around a first or central rotational axis 142. In at least one example embodiment, the bioreactor 100 may be rotated in alternating fashion, including, for example, in a first clockwise direction and then in a second counterclockwise direction around the central axis 142.

Although not illustrated, it should be recognized that, in various other example embodiments, a second rotational rocking component may be configured to move the bioreactor 100 about a second rotational axis 144 that passes through a center point of the bioreactor 100 normal to the central axis 142. In at least one example embodiment, the bioreactor 100 may be rotated in alternating fashion, including, for example, in a first clockwise direction and then in a second counterclockwise direction around the second axis 144. In at least one example embodiment, the bioreactor 100 may also be rotated around the second axis 144 and positioned in a horizontal or vertical orientation relative to gravity. The lateral rocking component 140 may be laterally associated with the bioreactor 100. For example, a plane of the lateral rocking component 140 may move laterally in the x-direction and y-direction.

The rotational and/or lateral movement of the bioreactor 100 may reduce the settling of cells and the likelihood of cells becoming trapped within a portion of the bioreactor 100. In at least one example embodiment, the rate of cells settling in the cell growth chamber 100 may be proportional to the density difference between the cells and the suspension media, according to Stoke's Law. In at least one example embodiment, a 180-degree rotation (fast) with a pause (having, for example, a total combined time of 30 seconds) repeated as described above may help to keep a population of suspended cells (for example, induced pluripotent stem cells prior to attachment). A minimum rotation of about 180-degrees may be preferred, however various degrees of rotation, including up to or greater than 360-degrees, may be used. Different rocking components may be used separately or may be combined in any combination. For example, a rocking component that rotates bioreactor 100 around central axis 142 may be combined with the rocking component that rotates bioreactor 100 around axis 144. Likewise, clockwise and counterclockwise rotation around different axes may be performed independently in any combination.

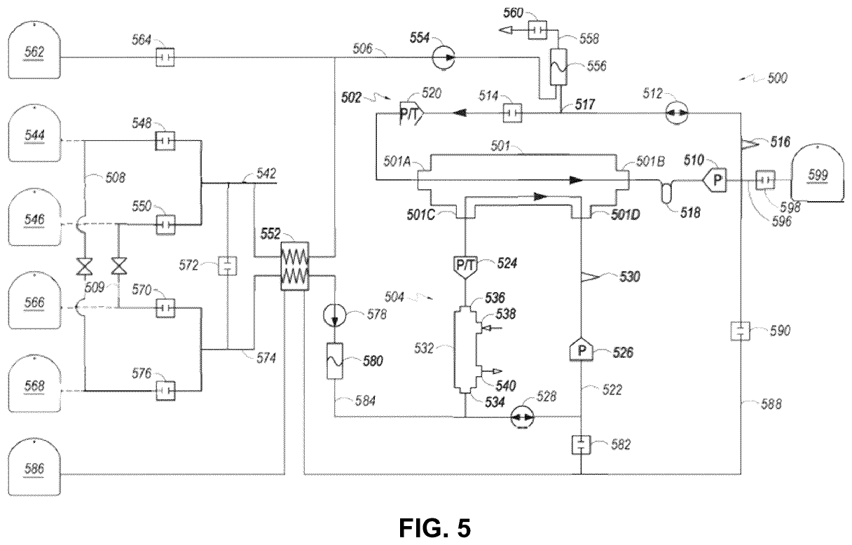

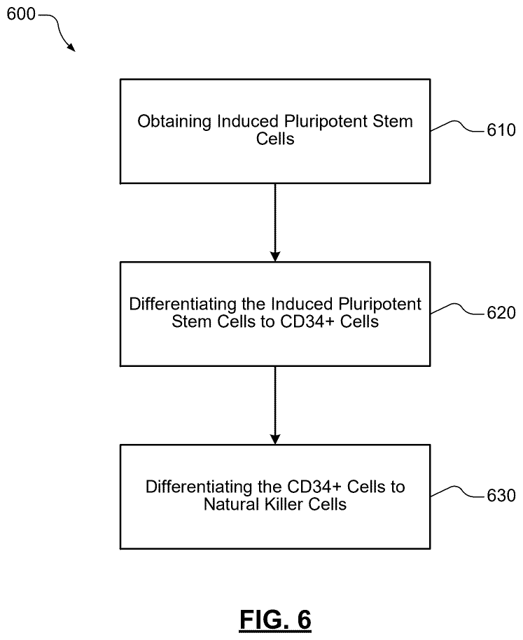

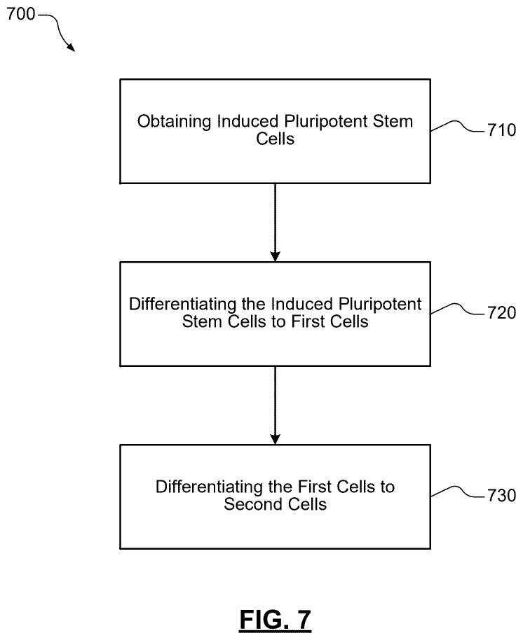

FIG. 5 is a schematic of an example cell expansion system 500, which may be like the cell expansion system 100 illustrated in FIG. 1, that illustrates example flow paths. In at least one example embodiment, the cells (for example, induced pluripotent stem cells) may be positioned in the intracapillary space, while a cell culture medium may be pumped through the extracapillary space to deliver nutrients to the cells via hollow fiber membrane perfusion during expansion. It should be recognized, however, in at least one other example embodiment, cells (for example, induced pluripotent stem cells) can be positioned in the extracapillary space, while the cell culture medium may be pumped through the intracapillary space to deliver nutrients to the cells via hollow fiber membrane perfusion during expansion. In at least one other example embodiment, cells (for example, induced pluripotent stem cells) may be positioned in the intracapillary space, while the cell culture medium may be pumped through both the extracapillary space and the intracapillary space.

As illustrated, the cell expansion system 500 may include a first fluid circulation path 502 (also referred to as the “intracapillary loop” or “IC loop”) and a second fluid circulation path 504 (also referred to as the “extracapillary loop” or “EC loop”). The first fluid flow path 506 may be fluidly associated with a cell growth chamber (also referred to as a “bioreactor”) 501 to form first fluid circulation path 502. The cell growth chamber 501 may be used as the hollow fiber cell growth chamber 24 of the cell expansion system 10 illustrated in FIG. 1. The cell growth chamber 501 may be used as the hollow fiber cell growth chamber 24 illustrated in FIG. 1 and/or the hollow fiber cell growth chamber 100 illustrated in FIG. 2 and/or the hollow fiber cell growth chamber 300 illustrated in FIG. 3. A first fluid may flow into cell growth chamber 501 through an intracapillary inlet port 501A. The first fluid may exit the cell growth chamber via an intracapillary outlet port 501B. In at least one example embodiment, the first fluid circulation path 502 may include a pressure gauge 510 configured to measure a pressure of the first fluid leaving the cell growth chamber 501. In at least one example embodiment, the first fluid circulation path 502 may include an intracapillary circulation pump 512 configured to control a first fluid flow rate. For example, the intracapillary circulation pump 512 may be configured to pump the first fluid in a first direction or a second direction that is opposite to the first direction. In the later instance, the intracapillary outlet port 501B may be used as an inlet, and the intracapillary inlet port 501A as an outlet. In at least one example embodiment, the first fluid circulation path 502 may include a sample port 516 and/or sample coil 518 configured for first fluid sample extraction. In at least one example embodiment, the first fluid circulation path 502 may include a pressure/temperature gauge 520 configured to detect the pressure and/or temperature of the first fluid during operation. In at least one example embodiment, the first fluid may enter the intracapillary loop 502 via valve 514. In at least one example embodiment, a portion of the cells may be flushed from the intracapillary loop 502 into a harvest bag 599, for example, via valve 598. It should be recognized that, in at least one other example embodiment, the first fluid circulation path 502 may include additional or fewer valves, pressure gauges, pressure sensors, temperature sensors, ports, and/or other devices disposed to isolate and/or measure characteristics of the first fluid along portions of the intracapillary loop 502.

A second fluid may flow into cell growth chamber 501 through an extracapillary inlet port 501C. The second fluid may leave the cell growth chamber 501 via an extracapillary outlet port 501D. In at least one example embodiment, the second fluid in the extracapillary loop 504 may contact an exterior facing surface of hollow fibers disposed in the cell growth chamber 501 thereby allowing diffusion of small molecules into and out of the hollow fibers. In at least one example embodiment, the extracapillary loop 504 may include a pressure/temperature gauge 524 configured to measure a pressure and/or temperature of the second fluid before the second fluid enters the cell growth chamber 501. In at least one example embodiment, the extracapillary loop 504 may include a pressure gauge 526 that is configured to measure a pressure of the second fluid, for example, as it leaves the cell growth chamber 501. In at least one example embodiment, the extracapillary loop 504 may include a sample port 530 configured for second fluid sample extraction.

In at least one example embodiment, the extracapillary loop 504 may include an extracapillary circulation pump 528 and an oxygenator or gas transfer module 532. For example, after leaving the cell growth chamber 501, the second fluid may pass through the extracapillary circulation pump 528 and to and through the oxygenator or gas transfer module 532. In at least one example embodiment, the extracapillary circulation pump 528 may be configured to control a second fluid flow rate. For example, like the intracapillary circulation pump 512, the extracapillary circulation pump 528 may be configured to pump the second fluid in a first direction or a second direction that is opposite to the first direction. In the later instance, the extracapillary outlet port 501D may be used as inlet, and the extracapillary inlet port 501C as an outlet.

In at least one example embodiment, the second fluid flow path 522 may be fluidly associated with the oxygenator or gas transfer module 532 via an oxygenator inlet port 534 and an oxygenator outlet port 536. For example, the second fluid may flow into the oxygenator or gas transfer module 532 via the oxygenator inlet port 534 and may leave or exit the oxygenator or gas transfer module 532 via the oxygenator outlet port 536. In at least one example embodiment, the oxygenator or gas transfer module 532 may be configured to add oxygen to and/or remove bubbles from the second fluid. For example, air and/or gas may flow into the oxygenator or gas transfer module 532 via a first filter 538 and may leave or exit (i.e., flow out of) the oxygenator or gas transfer device 532 through a second filter 540. The first and second filters 538, 540 may be configured to reduce or prevent contaminants from entering the oxygenator or gas transfer module 532. The second fluid in the second fluid circulation path 504 may be in equilibrium with gas entering the oxygenator or gas transfer module 532. In at least one example embodiment, air and/or gas may be purged from the cell expansion system 500, for example, during a priming sequence, air and/or gas may be vented to the atmosphere via the oxygenator or gas transfer module 532. It should be recognized that, in at least one other example embodiment, a second fluid circulation path 504 may include additional or fewer valves, pressure gauges, pressure sensors, temperature sensors, ports, and/or other devices disposed to isolate and/or measure characteristics of the second fluid along portions of the extracapillary loop 504.

In at least one example embodiment, an air removal chamber (ARC) 556 may be fluidly associated with the first circulation path 502. The air removal chamber 556 may include one or more ultrasonic sensors. For example, the air removal chamber 556 may include upper sensor and/or lower sensor which are configured to detect air and/or a lack of fluid and/or gas-fluid interface at certain measuring positions within the air removal chamber 556. The upper sensor may be disposed near a first end (e.g., top) of the air removal chamber 556. The lower sensor may be disposed near a second end (e.g., bottom) of the air removal chamber 556. Although ultrasonic sensors are discussed, it should be appreciated that, in various example embodiments, the air removal chamber 556 may include, additionally, or alternatively, one or more other sensors, including, for example, optical sensors. Air and/or gas purged from the cell expansion system 500 during portions of a priming sequence and/or other protocols may vent to the atmosphere out air valve 560 via line 558 that may be fluidly associated with air removal chamber 556.

In at least one example embodiment, the first fluid may include cells for example, from a first fluid container (which can also be referred to as a first media bag or a first bag) 562 and also fluid media (e.g., intracapillary media or fluid) from a second fluid container (which can also be referred to as a second media bag or a second bag) 546. Materials (e.g., cells and/or intracapillary media) form the first and second fluid containers 562, 546 may enter the first fluid circulation path 502 via a first fluid flow path 506. The first fluid container 562 may be fluidly associated with the first fluid flow path 506 and the first fluid circulation path 502 via valve 564. In at least one example embodiment, the second fluid container 546 and a third fluid container (which can also be referred to as a third media bag or third bag) 544 may be fluidly associated with the first fluid inlet path 542, for example, via valves 548 and 550, respectively, or with a second fluid inlet path 574, for example, via valves 570 and 576, respectively. In at least one example embodiment, the materials from the second fluid container 546 and/or the third fluid container 544 may be in fluid communication with a first sterile sealable input priming path 508 and/or a second sterile sealable input priming path 509.

In at least one example embodiment, a fourth fluid container (which can also be referred to as a fourth media bag or a fourth bag) 568 may include an extracapillary media, and a fifth fluid container (which can also be referred to a fifth media bag or a fifth bag) 566 may include a wash solution. Materials (i.e., extracapillary media and/or wash solution) from the fourth and fifth fluid containers 568, 566 may enter the first fluid circulation path 502 and/or the second fluid circulation path 504. For example, in at least one example embodiment, the fifth fluid container 566 may be fluidly associated with valve 570, where valve 570 is fluidly associated with first fluid circulation path 502, for example, via a distribution valve 572 and a first fluid inlet path 542. In at least one example embodiment, the fifth fluid container 566 may be fluidly associated with the second fluid circulation path 504 via the second fluid inlet path 574 and an extracapillary inlet path 584, for example, by opening valve 570 and closing distribution valve 572. The fourth fluid container 568 may be fluidly associated with valve 576, where valve 576 is fluidly associated with first fluid circulation path 502, for example, via the first fluid inlet path 542 and the distribution valve 572. In at least one example embodiment, the fourth fluid container 568 may be fluidly associated with the second fluid inlet path 574 by opening valve 576 and closing the distribution valve 572. In at least one example embodiment, the first fluid inlet path 542 and/or the second fluid inlet path 574 may be fluidly associated with an optional heat exchanger 552.

In at least one example embodiment, fluid may be advanced to the intracapillary loop 502 from the first fluid inlet path 542 and/or the second fluid inlet path 574 via an intracapillary inlet pump 554, and fluid may be advanced to the extracapillary loop 504 via an extracapillary inlet pump 578. In at least one example embodiment, an air detector 580 may also be associated with the extracapillary inlet path 584. The air detector 580 may include, for example, an ultrasonic sensor. In at least one example embodiment, the first and second fluid circulation paths 502, 504 may be fluidly associated with a waste line 588. For example, when valve 590 is in an open state or position, the intracapillary media may flow through the waste line 588 to a waste bag (also referred to as an outlet bag) 586. When valve 582 is opened, extracapillary media may flow through the waste line 588 to the waste bag 586. In at least one example embodiment, cells may be harvested, for example, via a cell harvest path 596. For example, cells from the cell growth chamber 501 may be harvested by pumping the intracapillary media containing the cells through the cell harvest path 596 and also valve 598 to a cell harvest bag 599.

In at least one example embodiment, as illustrated, the fluid in the first fluid circulation path 502 and second fluid circulation path 504 flows through cell growth chamber 501 in the same direction (i.e., a co-current configuration). Although not illustrated, it should be recognized that, in various other example embodiments, the cell expansion system 500 may also be configured to flow in a counter-current conformation. As illustrated in FIG. 5, fluid in the first fluid circulation path 502 may enter the bioreactor 501 at the intracapillary inlet port 501A and may leave or exit the bioreactor 501 at the intracapillary outlet port 501B. In at least one example embodiment, the first fluid flow path 506 may be fluidly connected to the first fluid circulation path 502, for example, via connection 517. Connection 517 may be a point or location from which the fluid may flow in opposite directions, for example, based on the direction and flow rates of the intracapillary inlet pump 554 and fluid circulation pump 512. Connection 517 may include any type of fitting, coupling, fusion, pathway, and/or tubing that allows the first fluid flow path 506 to be fluidly associated with the first fluid circulation path 502. In at least one example embodiment, connection 517 may include a T-fitting or coupling and/or a Y-fitting or coupling.

In at least one example embodiment, one or more of the gauges (e.g., pressure gauge 510, pressure/temperature gauge 520, pressure/temperature gauge 524, and/or pressure gauge 526), one or more of the valves (e.g., valve 514, valves 548, valves 550, valve 560, valve 564, valve 570, valve 572, valve 576, valve 582, valve 590, valve 596, and/or valve 598), one or more of the ports (e.g., intracapillary inlet port 501A, intracapillary outlet port 501B, extracapillary inlet port 501C, extracapillary outlet port 501D, sample port 516, sample port 530, oxygenator inlet port 534, and/or an oxygenator outlet port 536), one or more of the pumps (e.g., intracapillary circulation pump 512, extracapillary circulation pump 528, intracapillary inlet pump 554, and/or extracapillary inlet pump 578), one or more of the filters (e.g., first filter 538 and/or second filter 540), one or more coils (e.g., sample coil 518), one or more modules (e.g., oxygenator or gas transfer module 532), and/or one or more other components of the cell expansion system 500 may be in electrical communication with a control system (not shown). The control system may include a plurality of nodes, which can include various hardware, firmware, and/or software configured to control and/or communicate with the mechanical, electromechanical, and electrical components of the cell expansion system 500, including for example, a controller and a memory.

The controller (which can also be referred to as a processor), can be of any type of microcontroller, microprocessor, Field Programmable Gate Array (FPGA), Application Specific Integrated Circuit (ASIC), etc. An example controller may be the NK10DN512VOK10 microcontroller, made and sold by N9P USA, Incorporated, which is a microcontroller unit with a 32-bit architecture. Other examples controllers may include, for example, at least one of Qualcomm® Snapdragon® 800 and 801, Qualcomm® Snapdragon® 610 and 615 with 4G LTE Integration and 64-bit computing, Apple® A7 processor with 64-bit architecture, Apple® M7 motion coprocessors, Samsung® Exynos® series, the Intel® Core™ family of processors, the Intel® Xeon® family of processors, the Intel® Atom™ family of processors, the Intel Itanium® family of processors, Intel® Core® i5-4670K and i7-4770K 22 nm Haswell, Intel® Core® i5-3570K 22 nm Ivy Bridge, the AMD® FX™ family of processors, AMD® FX-4300, FX-6300, and FX-8350 32 nm Vishera, AMD® Kaveri processors, ARM® Cortex™-M processors, ARM® Cortex-A and ARM926EJ-S™ processors, other industry-equivalent processors, and may perform computational functions using any known or future-developed standard, instruction set, libraries, and/or architecture. The memory can be any type of memory including random access memory (RAM), read only memory (ROM), electrically erasable programmable ROM (EEPROM), a portable compact disc read-only memory (CD-ROM), an optical storage device, a magnetic storage device, any suitable combination of the foregoing, or other type of storage or memory device that stores and provides instructions to program and control the controller.

In various aspects, the present disclosure provides methods for preparing a bioreactor-based system (including, for example, the bioreactor illustrated in FIG. 1 and/or the bioreactor illustrated in FIG. 2 and/or the bioreactor illustrated in FIG. 3 and/or the bioreactor illustrated in FIG. 5) for differentiation of a cell population including induced pluripotent stem cells. In at least one example embodiment, preparing the bioreactor-based system for differentiation includes coating the bioreactor-based systems, and more specifically, at least a portion of the intracapillary spaces, and additionally, or alternatively, the extracapillary spaces, of the bioreactor-based systems. The intracapillary space, and additionally, or alternatively, the extracapillary space, of the bioreactor-based system may be coated using an active coating method, such as detailed, for example, in U.S. application Ser. No. 18/368,879 as filed Sep. 15, 2023 and titled EXPANDING CELLS and/or U.S. App. No. 63/538,610 as filed Sep. 15, 2023 and titled SYSTEMS AND METHODS FOR PRODUCING CHIMERIC ANTIGEN RECEPTOR CELLS and/or U.S. App. No. 63/461,989 as filed Apr. 26, 2023 and titled METHODS TO REMOVE SOLUTES WITHOUT SUSPENSION CELL LOSS and/or U.S. Pat. No. 11,702,634 as issued Jul. 18, 2023 and titled EXPANDING CELLS IN A BIOREACTOR, which was filed as U.S. application Ser. No. 15/943,536 on Apr. 2, 2018, and/or U.S. Pat. No. 10,577,575 as issued Mar. 3, 2020 and titled COATING A BIOREACTOR, which was filed as U.S. application Ser. No. 15/616,745 on Jun. 7, 2017, and/or U.S. Pat. No. 11,685,883 as issued Jun. 27, 2023 and titled METHODS AND SYSTEMS FOR COATING A CELL GROWTH SURFACE, which was filed as U.S. application Ser. No. 15/616,635 on Jun. 7, 2017, the entire disclosures of which are hereby incorporated by reference. In at least one example embodiment, the intracapillary space, and additionally, or alternatively, the extracapillary space, of the bioreactor-based system may be coated using coating reagents like laminin, vitronectin, or a combination of laminin and vitronectin.

In various aspects, the present disclosure provides methods for differentiation a cell population including induced pluripotent stem cells using bioreactor-based systems (including, for example, the bioreactor illustrated in FIG. 1 and/or the bioreactor illustrated in FIG. 2 and/or the bioreactor illustrated in FIG. 3 and/or the bioreactor illustrated in FIG. 5). The induced pluripotent stem cells can be either expanded in the bioreactor-based systems prior to differentiation or introduced into the bioreactor-based systems in appropriate numbers. In at least one example embodiment, the bioreactor-based systems may be expanded in the bioreactor-based systems using methods like those detailed in U.S. App. No. 63/595,366 as filed Nov. 2, 2023 and titled LOADING, DISTRIBUTING, AND EXPANDING CELLS IN A HOLLOW FIBER BIOREACTOR, the entire disclosure of which is hereby incorporated by reference. In at least one example embodiment, the induced pluripotent stem cells can be either adhered to the membrane of the intracapillary side or suspended in the fluid contained within the intracapillary lumen for differentiation. In each instance, the bioreactor-based systems provide unique environments that are conducive to the differentiation of induced pluripotent stem cells into other types of cells (e.g., natural killer cells, cardiomyocytes, mesenchymal stem cells, T cells, or any combination thereof) at least in part because of the surface area to volume rations and also the concentration effects imparted by the structure of the bioreactor-based systems. The bioreactor-based systems environment may increase the efficiency of the differentiation process enabling more completely differentiated cell product and/or a reduction in the time required for differentiation.

In various aspects, the present disclosure provides methods for differentiating a cell population including induced pluripotent stem cells using bioreactor-based systems (including, for example, the bioreactor illustrated in FIG. 1 and/or the bioreactor illustrated in FIG. 2 and/or the bioreactor illustrated in FIG. 3 and/or the bioreactor illustrated in FIG. 5) to form natural killer cells.

FIG. 5 illustrates an example method 600 for differentiating a cell population including induced pluripotent stem cells using a bioreactor-based system to form natural killer cells.

The method includes obtaining 610 induced pluripotent stem cells. In at least one example embodiment, the induced pluripotent stem cells may be obtained by expanding the induced pluripotent stem cells using the bioreactor-based system. In other example embodiments, the induced pluripotent stem cells may be obtained by introducing the induced pluripotent stem cells into the bioreactor-based system.

Once the induced pluripotent stem cells are obtained, the method 600 includes differentiating 620 the induced pluripotent stem cells to CD34+ cells, which are an intermediary precursor cell type on the path to nature killer cell differentiation. Once the CD34+ cells are obtained, the method 600 may include differentiating 630 the CD34+ cells to natural killer cells. CD34+ cells are a necessary intermediary on the differentiation pathway from induced pluripotent stem cells to natural cell. CD34+ cells are also known as hematopoietic stem cells and are the subtype of cell from which many immune cells are derived in natural systems.

In at least one example embodiment, differentiating 620 the induced pluripotent stem cells to CD34+ cells may include using a pump in the bioreactor-based system to initiate a replacement of induced pluripotent stem cells expansion medium with medium formulated to induce the development of CD34+ hematopoietic stem cells. After an appropriate amount of time has passed, which may be determined or defined by the presence of fully differentiated CD34+ cells taken from the bioreactor-based system, a coating/cell binding reagent may be introduced. The coating/cell binding reagent may stimulate membrane bound integrin proteins, as well as bind to available surface area on the hollow-fiber membrane to provide stimulation over time may occur, which can facilitate the transition 630 from CD34+ cells to natural killer cells. A second replacement of the bioreactor-based system medium may occur at this point to replace medium that is relevant to CD34+ expansion with a differently formulate medium aimed at the directed expansion of natural killer cells. Each of the three cell types represented here (iPSC, CD34+, and NK) may require different culture methods and media. The bioreactor-based system may provide a range of culture methods that may be appropriate to each of the three cell types described in the order described.

In various aspects, the present disclosure provides methods for differentiating a cell population including induced pluripotent stem cells using bioreactor-based systems (including, for example, the bioreactor illustrated in FIG. 1 and/or the bioreactor illustrated in FIG. 2 and/or the bioreactor illustrated in FIG. 3 and/or the bioreactor illustrated in FIG. 5) to form a different type of cell such as natural killer cells, cardiomyocytes, mesenchymal stem cells, T cells, neuronal cells, cell precursors, liver cells, lung cells, pancreatic cells, skin cell fibroblasts, kidney cells, skeletal muscle cells, smooth muscle cells, red blood cells, megakaryocytes or any subset of the ectoderm, mesoderm, or endoderm germ layers of tissue.

FIG. 7 illustrates an example method 700 for differentiating a cell population including induced pluripotent stem cells using a bioreactor-based system to form a second type of cell.

The method includes obtaining 710 induced pluripotent stem cells. In at least one example embodiment, the induced pluripotent stem cells may be obtained by expanding the induced pluripotent stem cells using the bioreactor-based system. In other example embodiments, the induced pluripotent stem cells may be obtained by introducing the induced pluripotent stem cells into the bioreactor-based system.

Once the induced pluripotent stem cells are obtained, the method 700 includes differentiating 720 the induced pluripotent stem cells to a first type of cells. In at least one example embodiment the first type of cells may be CD34+ cells and/or any subset of the ectoderm, mesoderm, or endoderm germ layers of tissue, which are an intermediary precursor cell type on the path to nature killer cell differentiation. Once the first type of cells are obtained, the method 700 may include differentiating 730 the first type of cells to a second type of cells. In at least one example embodiment, the first type of cell, such as the CD34+ cells, are a necessary intermediary on the differentiation pathway from induced pluripotent stem cells to the second type of cells.

In at least one example embodiment, differentiating 720 the induced pluripotent stem cells to the first type of cells may include using a pump in the bioreactor-based system to initiate a replacement of induced pluripotent stem cells expansion medium with medium formulated to induce the development of the first type of cells. After an appropriate amount of time has passed, which may be determined or defined by the presence of fully differentiated first type of cell taken from the bioreactor-based system, a coating/cell binding reagent may be introduced. The coating/cell binding reagent may stimulate membrane bound integrin proteins, as well as bind to available surface area on the hollow-fiber membrane to provide stimulation over time may occur, which can facilitate the transition 730 from the first type of cells to the second type of cells. A second replacement of the bioreactor-based system medium may occur at this point to replace medium that is relevant to first type of cell expansion with a differently formulate medium aimed at the directed expansion of the second type of cells. Each of the three cell types represented here (iPSC, first type of cells, and second type of sells) may require different culture methods and media. The bioreactor-based system may provide a range of culture methods that may be appropriate to each of the three cell types described in the order described.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A method of differentiating induced pluripotent stem cells using a hollow fiber bioreactor, wherein the method comprises:
obtaining induced pluripotent stem cells in the hollow fiber bioreactor; and
moving a differentiating medium through the hollow fiber bioreactor.

2. The method of claim 1, wherein the obtaining of the induced pluripotent stem cells includes expanding the induced pluripotent stem cells using the hollow fiber bioreactor.

3. The method of claim 1, wherein the obtaining of the induced pluripotent stem cells includes introducing the induced pluripotent stem cells into the hollow fiber bioreactor.

4. A method of preparing natural killer cells using a hollow fiber bioreactor, the method comprising:
obtaining induced pluripotent stem cells;
differentiating the induced pluripotent stem cells to CD34+ cells using the hollow fiber bioreactor; and
differentiating the CD34+ cells to natural killer cells using the hollow fiber bioreactor.

5. The method of claim 4, wherein the obtaining of the induced pluripotent stem cells includes expanding the induced pluripotent stem cells using the hollow fiber bioreactor.

6. The method of claim 4, wherein the obtaining of the induced pluripotent stem cells includes introducing the induced pluripotent stem cells into the hollow fiber bioreactor.

7. A system for cell expansion, the system including
a hollow fiber cell growth chamber including a hollow fiber membrane;
a first circulation path in fluid communication with the hollow fiber cell growth chamber; and
a second circulation path in fluid communication with the hollow fiber cell growth chamber.

8. The system of claim 7, wherein the first circulation path includes a first fluid flow path with a first end in communication with a first inlet of the hollow fiber cell growth chamber and a second end in communication with a first outlet of the hollow fiber cell growth chamber.

9. The system of claim 7, wherein the second circulation path includes a second fluid flow path with a first end in communication with an inlet port of the hollow fiber cell growth chamber and a second end in communication with an outlet port of the hollow fiber cell growth chamber.

10. The system of claim 9, wherein fluid in the second fluid flow path is in fluid communication with an outside of the hollow fiber membrane.

11. The system of claim 7, further comprising:
a first fluid flow device configured to control a first flow of fluid in the first circulation path; and
a second fluid flow device configured to control a second flow of fluid in the second circulation path.

12. The system of claim 7, wherein the hollow fiber membrane includes an intracapillary portion and an extracapillary portion.

13. The system of claim 12, wherein the intracapillary portion is configured to enable expansion of induced pluripotent stem cells.

14. The system of claim 13, wherein the extracapillary portion is configured to deliver nutrients from a cell culture medium to the induced pluripotent stem cells via hollow fiber membrane profusion during the expansion.

15. The system of claim 13, wherein the intracapillary portion is configured to deliver nutrients from a cell culture medium to the induced pluripotent stem cells during the expansion.

16. The system of claim 13, wherein the extracapillary portion is configured to deliver nutrients from a cell culture medium to the induced pluripotent stem cells via hollow fiber membrane profusion during the expansion and the intracapillary portion is configured to deliver nutrients from the cell culture medium to the induced pluripotent stem cells during the expansion.
